(19) United States
(12) Patent Application Publication
(10) Pub. No.: US 2021/0220096 A1
(43) Pub. Date: Jul. 22, 2021

(54) VISUAL DENTAL IRRIGATOR

(71) Applicant: BANGBANG YOUPIN (SHENZHEN) INDUSTRIAL CO., LTD., SHENZHEN CITY (CN)

(72) Inventor: WENMING LI, SHENZHEN CITY (CN)

(21) Appl. No.: 17/105,970

(22) Filed: Nov. 27, 2020

(30) Foreign Application Priority Data
Jan. 16, 2020 (CN) 202020101862.4

(51) Int. Cl. A61C 17/02; A61C 17/16

(52) U.S. Cl. CPC A61C 17/02; A61C 17/16

(57) ABSTRACT

A visual dental irrigator relates to the technical field of dental cleaning equipment, including: a casing; a water tank detachably mounted on one end of the casing; a cleaning head mounted on the other end of the casing; a camera mounted on one end of the casing adjacent to the cleaning head; a flow amount control assembly provided inside the casing and connected between the cleaning head and the water tank; a control motherboard electrically connected to the camera and the flow amount control assembly; a communication module provided on the control motherboard for information transmission; and an operation button and a power source electrically connected to the control motherboard. The above technical solution has the advantages of assisting oral observation, effectively cleaning the oral cavity, and improving the dental cleaning experience.

### CROSS-REFERENCE TO RELATED PATENT APPLICATION

This application claims the benefit of priority to China Patent Application No. 202020101862.4, filed on Jan. 16, 2020 in People's Republic of China. The entire content of the above identified application is incorporated herein by reference.

### FIELD OF THE DISCLOSURE

The present disclosure relates to the technical field of dental cleaning equipment, in particular to a visual dental irrigator.

### BACKGROUND OF THE DISCLOSURE

The dental irrigator is special equipment for washing teeth, and is a cleaning appliance for removing calculus, smoke, plaque, and the like. Generally, the dental irrigator includes an interconnected handle and a working head with a working knife tip. The handle is provided with a transducer for driving the working head to vibrate, and a fluid passage communicated with the working head.

The existing dental irrigators are mainly divided into two types: medical scalers and household scalers. Medical scalers generally rely on the dentist's manual operation. However, this method of tooth cleaning has many disadvantages. For example, dentists need to spend a lot of physical energy during tooth washing, and have to keep their heads down for a long time to make the oral observation, which causes the dentist to work easily for a long time and reduce the quality of the operation. As to household dental irrigators, they are usually used to perform tooth washing by the user him/herself. No matter which type of dental irrigators is used, the cleaning condition is observed with the naked eyes; however the light in the mouth is dark, so users usually rely on external light or personal experience to observe the cleaning condition, thus resulting in inaccurate judgments and insufficient cleaning

### SUMMARY OF THE DISCLOSURE

The primary purpose of the present disclosure is to provide a visual dental irrigator, which has the advantages of assisting oral observation, effectively cleaning the oral cavity, and improving the dental cleaning experience.

To achieve the above purpose, the technical solution adopted by the present disclosure is to provide a visual dental irrigator, including: a casing; a water tank detachably mounted on one end of the casing; a cleaning head mounted on the other end of the casing; a camera mounted on one end of the casing adjacent to the cleaning head; a flow amount control assembly provided inside the casing and connected between the cleaning head and the water tank; a control motherboard electrically connected to the camera and the flow amount control assembly; a communication module provided on the control motherboard for information transmission; and an operation button and a power source electrically connected to the control motherboard.

In the present disclosure, the flow amount control assembly includes: a connecting cavity provided in the casing; a water inlet pipe assembled on a water inlet end of the connecting cavity and connected to the water tank; a water outlet pipe assembled on a water outlet end of the connecting cavity and connected to the cleaning head; and a driving assembly assembled at one end of the connecting cavity, wherein a water inlet check valve is provided at the water inlet end, and a water outlet check valve is provided at the water outlet end.

In the present disclosure, the driving assembly includes: a driving motor mounted in the casing; a driving gear provided on an output shaft of the driving motor; a gear seat rotatably mounted on the casing and engaging with the driving gear; a piston rod eccentrically mounted on the gear seat; and a piston body slidingly mounted in the connecting cavity and sleeved on the piston rod, wherein a limiting hole is provided at an end of the piston rod, and a limiting pin assembled with the piston body is inserted through the limiting hole.

In the present disclosure, the casing includes: a first fixing member and a second fixing member provided in the casing for assembling the driving assembly and the connecting cavity.

In the present disclosure, the visual dental irrigator further includes: a first connecting member disposed between the casing and the water tank; and a second connecting member provided between the casing and the cleaning head, wherein a first waterproof ring is provided on the first connecting member, and a second waterproof ring is provided on the second connecting member.

In the present disclosure, a gravity ball is provided at one end of the water inlet pipe.

In the present disclosure, an indicator light electrically connected to the control motherboard is further provided at one side of the casing.

In the present disclosure, a charging interface electrically connected to the power source is provided at one side of the casing, and a waterproof rubber plug is disposed on the charging interface.

After the above technical solution is adopted, the beneficial effect of the present disclosure is as follows.

1. In the present disclosure, a camera is mounted on the end of the casing adjacent to the cleaning head, and the shooting content is transmitted to the display device in real time by the communication module, so that the picture showing the oral cavity interior can be clearly captured during tooth washing, and some oral problems can be found in real time, which is convenient for dentists or users to intuitively learn about the oral condition from the display device, so as to improve the cleanliness of the teeth and the cleaning experience. In addition, the cleaning head is long and partially bent, so the camera is not easy to get water stains during the shooting process, thereby ensuring the shooting effect of the camera and improving the shooting resolution.

2. The visual dental irrigator of the present disclosure has a reasonable structural design. When in use, a driving motor drives the driving gear and the gear seat to rotate, thereby eccentrically driving the piston body to perform piston movement in the connecting cavity and realizing the control of the inflow and outflow of the connecting cavity to effectively increase the impact force of the water column and the flow amount, which further meets the needs of people for the use of dental irrigators.

### BRIEF DESCRIPTION OF THE DRAWINGS

In order to more clearly explain the embodiments of the present disclosure or the technical solutions in the related art, the drawings used in the description of the embodiments or the related art will be briefly introduced below. Obviously, the drawings in the following description are only some embodiments of the present disclosure. For those of ordinary skill in the art, other drawings can be obtained based on these drawings without paying creative labor.

FIG. 1 is a schematic structural diagram of the present embodiment.

FIG. 2 is an exploded view of the structure of the present embodiment.

FIG. 3 is a schematic diagram of a partial structure of the present embodiment.

Reference numeral: 1. casing; 11. first fixing member; 12. second fixing member; 2. water tank; 3. cleaning head; 4. camera; 5. flow amount control assembly; 6. control motherboard; 7. operation button; 8. power source; 51. connecting cavity; 52. water inlet pipe; 521. gravity ball; 53. water outlet pipe; 54. water inlet check valve; 55. water outlet check valve; 56. driving assembly; 561. driving motor; 563. gear seat; 564. piston rod; 565. piston body; 5641. limiting hole; 566. limiting pin; 13. first connecting member; 131. first waterproof ring; 14. second connecting member; 141. second waterproof ring; 9. indicator light; 10. charging interface.

### DETAILED DESCRIPTION OF THE EXEMPLARY EMBODIMENTS

The present disclosure will be further described in detail below with reference to the drawings.

The specific embodiment is only an explanation of the disclosure, and is not a limitation on the present disclosure. After reading this specification, those skilled in the art can make modifications that do not contribute to this embodiment as needed. All new claims are protected by patent law.

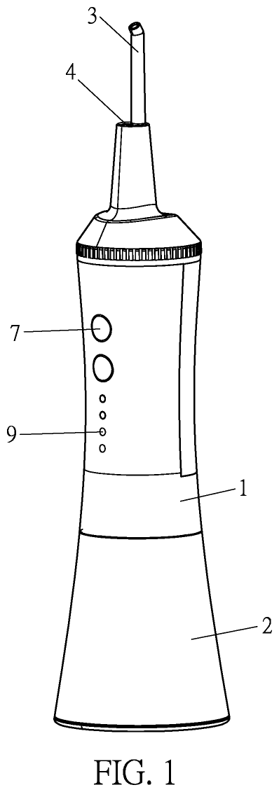

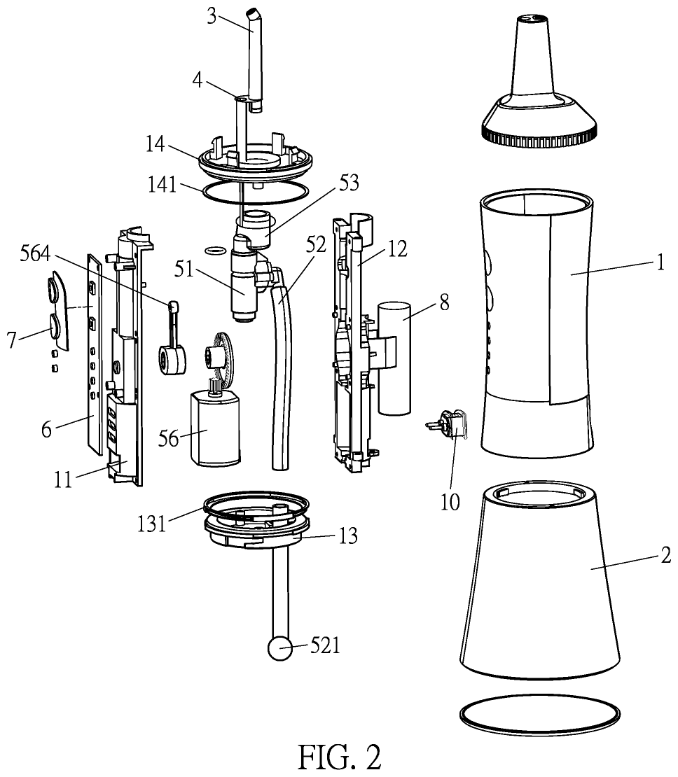

The embodiment relates to a visual dental irrigator, as shown in FIG. 1 and FIG. 2, including: a casing 1; a water tank 2 detachably mounted on one end of the casing 1; a cleaning head 3 mounted on the other end of the casing 1; a camera 4 mounted on one end of the casing 1 adjacent to the cleaning head 3; a flow amount control assembly 5 provided inside the casing 1 and connected between the cleaning head 3 and the water tank 2; a control motherboard 6 electrically connected to the camera 4 and the flow amount control assembly 5; a communication module provided on the control motherboard 6 for information transmission; and an operation button 7 and a power source 8 electrically connected to the control motherboard 6. When using the dental irrigator to wash teeth, the camera 4 at one end of the casing 1 can be used to capture the picture showing the oral cavity interior and play it on the display device in real time to assist the user or dentist in observing the oral cleaning condition, thereby assisting oral observation and improving the cleaning experience.

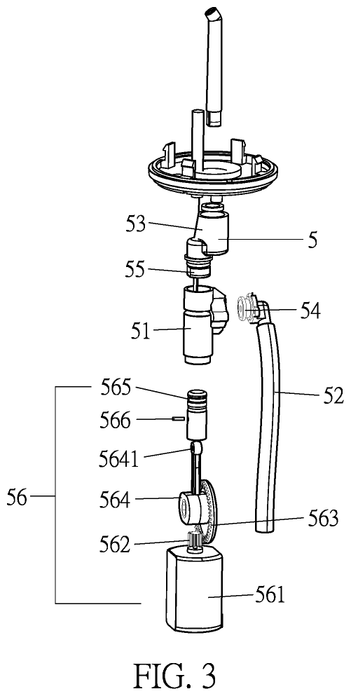

As shown in FIG. 2 and FIG. 3, the flow amount control assembly 5 includes: a connecting cavity 51 provided in the casing 1; a water inlet pipe 52 assembled on a water inlet end of the connecting cavity 51 and connected to the water tank 2; a water outlet pipe 53 assembled on a water outlet end of the connecting cavity 51 and connected to the cleaning head 3; and a driving assembly 56 assembled at one end of the connecting cavity 51. A gravity ball 521 is provided at one end of the water inlet pipe 52, thereby improving the water inlet effect at one end of the water inlet pipe 52. The driving assembly 56 controls the inflow and outflow of the connecting cavity 51 by adjusting the air pressure inside the connecting cavity 51. Under the action of the driving assembly 56, the water inlet pipe 52 can introduce the water in the water tank 2 into the chamber connected to the connecting cavity 51, and then spray it out from the water outlet pipe 53, so as to realize the outward spraying of the water column. The casing 1 is provided with a first fixing member 11 and a second fixing member 12 for assembling the driving assembly 56 and the connecting cavity 51, so as to enhance the structural stability inside the dental irrigator. In this embodiment, a water inlet check valve 54 is provided at the water inlet end, and a water outlet check valve 55 is provided at the water outlet end. When the flow amount control assembly 5 is in operation, water can only be introduced into the connecting cavity 51 from the water inlet pipe 52 and led out from the water outlet pipe 53 to ensure the normal flow amount of the waterway during the operation of the flow amount control assembly 5. The connecting cavity 51 is configured as a three-way structure.

As shown in FIG. 3, in the present embodiment, the driving assembly 56 comprises: a driving motor 561 mounted in the casing 1; a driving gear 562 provided on an output shaft of the driving motor 561; a gear seat 563 rotatably mounted on the casing 1 and engaging with the driving gear 562; a piston rod 564 eccentrically mounted on the gear seat 563; and a piston body 565 slidingly mounted in the connecting cavity 51 and sleeved on the piston rod 564. A limiting hole 5641 is provided at an end of the piston rod 564, and a limiting pin 566 assembled with the piston body 565 is inserted through the limiting hole 5641. The piston body 565 is provided with two sealing rings, thereby ensuring the air tightness in the connecting cavity 51. When the flow amount control assembly 5 is in operation, the driving motor 561 drives the gear seat 563 to rotate relative to the casing 1 through the driving gear 562. Since the piston rod 564 is eccentrically mounted on the gear seat 563, the rotating gear seat 563 can drive the piston rod 564 and the piston body 565 performs a piston movement relative to the connecting cavity 51, thereby adjusting the air pressure in the connecting cavity 51 and controlling the inflow and outflow of the connecting cavity 51.

Specifically, when the driving motor 561 drives the piston body 565 to extend out of the connecting cavity 51 through the driving gear 562 and the gear seat 563, the chamber of the connecting cavity 51 generates a certain suction force, so that the water inlet check valve 54 on the water inlet end opens and achieves water ingress; and when the piston body 565 retracts into the connecting cavity 51, the piston body 565 can push the water in the connecting cavity 51 to be discharged outward through the water outlet check valve 55, thereby realizing the ejection of the water column effect. The gear seat 563 is rotatably assembled on the first fixing member 11 and/or the second fixing member 12 along the axis, so that when the gear seat 563 is driven by the driving gear 562, it can rotate relative to the first fixing member 11 and/or the second fixing member 12. The overall structure design is reasonable. When in use, the driving motor 561 drives the driving gear 562 and the gear seat 563 to rotate, thereby eccentrically driving the piston body 565 to perform piston movement in the connecting cavity 51 and realizing the control of the inflow and outflow of the connecting cavity 51, so as to effectively increase the impact of the water column and the flow amount and further meet people's needs for the use of dental irrigators.

As shown in FIG. 2, the visual dental irrigator in the present embodiment further includes: a first connecting member 13 disposed between the casing 1 and the water tank 2; and a second connecting member 14 provided between the casing 1 and the cleaning head 3, wherein a first waterproof ring 131 is provided on the first connecting member 13, and a second waterproof ring 141 is provided on the second connecting member 14. By means of the manner, the various parts of the visual dental irrigator are assembled stably and sealed tightly, thereby effectively improving the service life. An indicator light 9 electrically connected to the control motherboard 6 is further provided at one side of the casing 1 to facilitate users to know the use status or power status of the visual dental irrigator in real time. A charging interface 10 electrically connected to the power source 8 is provided at one side of the casing 1, and a waterproof rubber plug is disposed on the charging interface 10. An operation button 7 and the waterproof rubber plug are made of soft silicone.

The working principle of the present disclosure is roughly as follows. In the present embodiment, the camera 4 is mounted on one end of the casing 1 adjacent to the cleaning head 3, and the shooting content is transmitted to the display device in real time by the communication module, so that the picture showing the oral cavity interior can be clearly captured during tooth washing, and some oral problems can be found in real time, which is convenient for dentists or users to intuitively learn about the oral condition from the display device, so as to improve the cleanliness of the teeth and the cleaning experience. In addition, the cleaning head 3 is long and partially bent, so the camera 4 is not easy to get water stains during the shooting process, thereby ensuring the shooting effect of the camera 4 and improving the shooting resolution.

The above is only used to explain the technical solution of the present disclosure and is not limitative. Other modifications or equivalent replacements made by those skilled in the art to the technical solution of the present disclosure are provided without departing from the spirit and scope of the technical solution of the present disclosure. It should be covered by the scope of claims of the present disclosure.

What is claimed is:

1. A visual dental irrigator, comprising:
a casing (1);
a water tank (2) detachably mounted on one end of the casing (1);
a cleaning head (3) mounted on the other end of the casing (1);
a camera (4) mounted on one end of the casing (1) adjacent to the cleaning head (3);
a flow amount control assembly (5) provided inside the casing (1) and connected between the cleaning head (3) and the water tank (2);
a control motherboard (6) electrically connected to the camera (4) and the flow amount control assembly (5);
a communication module provided on the control motherboard (6) for information transmission; and
an operation button (7) and a power source (8) electrically connected to the control motherboard (6).

2. The visual dental irrigator according claim 1, wherein the flow amount control assembly (5) comprises:
a connecting cavity (51) provided in the casing (1);
a water inlet pipe (52) assembled on a water inlet end of the connecting cavity (51) and connected to the water tank (2);
a water outlet pipe (53) assembled on a water outlet end of the connecting cavity (51) and connected to the cleaning head (3); and
a driving assembly (56) assembled at one end of the connecting cavity (51), wherein a water inlet check valve (54) is provided at the water inlet end, and a water outlet check valve (55) is provided at the water outlet end.

3. The visual dental irrigator according claim 2, wherein the driving assembly (56) comprises:
a driving motor (561) mounted in the casing (1);
a driving gear (562) provided on an output shaft of the driving motor (561);
a gear seat (563) rotatably mounted on the casing (1) and engaging with the driving gear (562);
a piston rod (564) eccentrically mounted on the gear seat (563); and
a piston body (565) slidingly mounted in the connecting cavity (51) and sleeved on the piston rod (564), wherein a limiting hole (5641) is provided at an end of the piston rod (564), and a limiting pin (566) assembled with the piston body (565) is inserted through the limiting hole (5641).

4. The visual dental irrigator according claim 2, wherein a first fixing member (11) and a second fixing member (12) are provided in the casing (1) for assembling the driving assembly (56) and the connecting cavity (51).

5. The visual dental irrigator according claim 1, further comprising:
a first connecting member (13) disposed between the casing (1) and the water tank (2); and a second connecting member (14) provided between the casing (1) and the cleaning head (3), wherein a first waterproof ring (131) is provided on the first connecting member (13), and a second waterproof ring (141) is provided on the second connecting member (14).

6. The visual dental irrigator according claim 2, wherein a gravity ball (521) is provided at one end of the water inlet pipe (52).

7. The visual dental irrigator according claim 1, wherein an indicator light (9) electrically connected to the control motherboard (6) is further provided at one side of the casing (1).

8. The visual dental irrigator according claim 1, wherein a charging interface (10) electrically connected to the power source (8) is provided at one side of the casing (1), and a waterproof rubber plug is disposed on the charging interface (10).
